(19) United States
(12) Patent Application Publication
(10) Pub. No.: US 2021/0158973 A1
(43) Pub. Date: May 27, 2021

(54) INTELLIGENT DATA ANALYSIS METHOD AND DEVICE, COMPUTER DEVICE, AND STORAGE MEDIUM

(71) Applicant: Ping An Technology (Shenzhen) Co., Ltd., Shenzhen (CN)

(72) Inventors: Xianxian CHEN, Shenzhen (CN); Xiaowen RUAN, Shenzhen (CN); Liang XU, Shenzhen (CN)

(73) Assignee: Ping An Technology (Shenzhen) Co., Ltd., Shenzhen (CN)

(21) Appl. No.: 17/168,925

(22) Filed: Feb. 5, 2021

Related U.S. Application Data
(63) Continuation of application No. PCT/CN2019/116942, filed on Nov. 11, 2019.

(30) Foreign Application Priority Data
Aug. 19, 2019 (CN) 201910763137.5

(51) Int. Cl. G16H 50/70; G06N 20/20

(52) U.S. Cl. CPC G16H 50/70; G06N 20/20

(57) ABSTRACT

The application discloses an intelligent data analysis method and device, a computer device, and a storage medium. The intelligent data analysis method includes that: a public opinion factor obtained and a public opinion index carrying a time label are taken as first portrait data (S40); original sample data is obtained based on the first portrait data and medical data; the original sample data is cleaned to obtain sample data to be processed (S50); lag processing is performed on the sample data to be processed to obtain lag sample data (S60); feature expansion is performed on the lag sample data to obtain target sample data (S70); and an improved multi-granularity cascading random forest algorithm is used to train the target sample data to obtain a target forecast model (S80); the improved multi-granularity cascading random forest algorithm includes a pooling layer, which is used for retaining data features (S90).

### CROSS-REFERENCE TO RELATED APPLICATIONS

The application is a continuation under 35 U.S.C. § 120 of PCT Application No. PCT/CN2019/116942 filed on Nov. 11, 2019, which claims priority under 35 U.S.C. § 119(a) and/or PCT Article 8 to Chinese Patent Application No. 201910763137.5, filed on Aug. 19, 2019, the disclosures of which are hereby incorporated by reference in their entireties.

### TECHNICAL FIELD

The application relates to the field of data forecast technology, in particular to an intelligent data analysis method and device, a computer device, and a storage medium.

### BACKGROUND

With the rapid development of the information age, data forecast technology is also developing continuously. At present, when major scientific research institutions make forecasts on medical data, the accuracy of model forecast is low due to the lag of some medical data. For example, for infectious diseases with a certain incubation period (such as chickenpox), when the conditions for an outbreak (such as temperature and humidity) are met, the outbreak may occur in the next period, which results in the low accuracy of model forecast. Thus, citizens cannot timely prevent diseases and the severity of the outbreak cannot be controlled.

### SUMMARY

Embodiments of the application provide an intelligent data analysis method and device, a computer device, and a storage medium.

An intelligent data analysis method includes the following operations.

According to preset keywords, a crawler tool is used to crawl public opinion data obtained by a third-party information platform.

At least one hit entry is determined based on the public opinion data, the hit entry corresponding to a public opinion factor.

Medical data in historical unit time and a public opinion index corresponding to the hit entry are obtained, the public opinion index carrying a time label.

The public opinion factor and the public opinion index carrying the time label are taken as first portrait data.

Original sample data is obtained based on the first portrait data and the medical data.

The original sample data is cleaned to obtain sample data to be processed.

Lag processing is performed on the sample data to be processed to obtain lag sample data.

Feature expansion is performed on the lag sample data to obtain target sample data.

An improved multi-granularity cascading random forest algorithm is used to train the target sample data to obtain a target forecast model. The improved multi-granularity cascading random forest algorithm includes a pooling layer which is used for retaining data features.

A computer device includes a memory, a processor, and a computer readable instruction stored in the memory and capable of running on the processor. The processor, when executing the computer readable instruction, implements the above steps of the intelligent data analysis method.

A readable storage medium stores a computer readable instruction. The computer readable instruction, when executed by the processor, implements the above steps of the intelligent data analysis method.

The details of one or more embodiments of the application are set out in the drawings and description below, and other features and advantages of the application will become apparent from the description, the drawings and the claims.

### BRIEF DESCRIPTION OF THE DRAWINGS

In order to more clearly illustrate technical solutions in embodiments of the application, the drawings needed in the description of the embodiments are simply introduced below. It is apparent for those of ordinary skill in the art that the accompanying drawings in the following description are only some embodiments of the application, and some other accompanying drawings may also be obtained according to these drawings on the premise of not contributing creative effort.

FIG. 1 is a schematic diagram of an application environment of an in the embodiments of the application according to an embodiment of the application.

FIG. 2 is a flowchart of an intelligent data analysis method according to an embodiment of the application.

FIG. 3 is a specific flowchart of S60 in FIG. 2.

FIG. 4 is a specific flowchart of S80 in FIG. 2.

FIG. 5 is a flowchart of an intelligent data analysis method according to an embodiment of the application.

FIG. 6 is a specific flowchart of S90 in FIG. 2.

FIG. 7 is a specific flowchart of S92 in FIG. 6.

FIG. 8 is a schematic diagram of an intelligent data analysis device according to an embodiment of the application.

FIG. 9 is a schematic diagram of a computer device according to an embodiment of the application.

### DETAILED DESCRIPTION OF THE EMBODIMENTS

The technical solutions in the embodiments of the application will be described clearly and completely below in combination with the drawings in the embodiments of the application. It is apparent that the described embodiments are not all but part of the embodiments of the application. All other embodiments obtained by those of ordinary skill in the art based on the embodiments in the application without creative work shall fall within the scope of protection of the application.

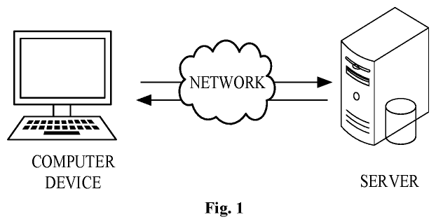

The intelligent data analysis method provided by the embodiments of the application may be applied to an intelligent data analysis tool. The intelligent data analysis tool may train different forecast models according to sample data corresponding to different themes (such as chickenpox and influenza), especially for the sample data with a lag, may effectively guarantee the accuracy of model forecast. The intelligent data analysis method may be applied in the application environment shown in FIG. 1. A computer device communicates with a server through a network. The computer device may be, but not limited to, a personal computer, a laptop, a smart phone, a tablet computer, and a portable wearable device. The server may be realized by an independent server.

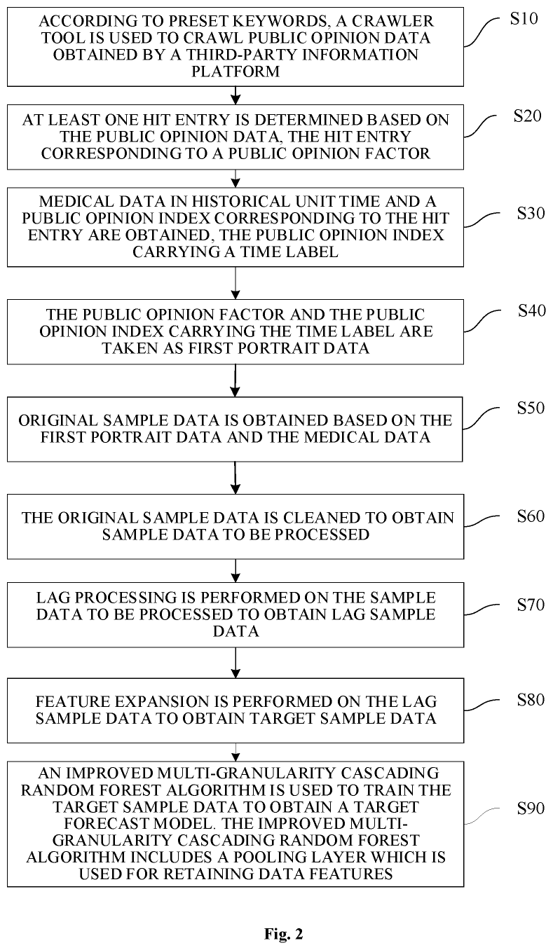

In an embodiment, as shown in FIG. 2, an intelligent data analysis method is provided. Illustrated by the application of the method to the server in FIG. 1, the method includes the following steps.

At S10, according to preset keywords, a crawler tool is used to crawl public opinion data obtained by a third-party information platform.

The preset keywords are some preset keywords related to communicable diseases, such as chickenpox, redness and swelling, itchy herpes, and water herpes. The public opinion data refers to text data publicly released by different users in the third-party information platform to reflect the occurrence of social events. Specifically, with the rapid development of the information age, users are more inclined to use various information platforms to query required information, such as whether they are suffering from diseases according to their own symptoms, and when a certain communicable disease (such as chickenpox) breaks out, there is bound to be more search traffic or attention. Therefore, in the embodiment, a crawler tool is also used to crawl the public opinion data including the preset keywords in the third-party information platform (such as Baidu, weibo, or WeChat) according to the preset keywords. It is to be noted that a part of default preset keywords of the preset keywords related to the communicable diseases in the embodiment may be set in advance, and then synonyms corresponding to the default keywords may be taken, so as to obtain more keywords for crawling and obtain more relevant information, which provides sufficient data sets for subsequent model training.

At S20, at least one hit entry is determined based on the public opinion data, the hit entry corresponding to a public opinion factor.

Specifically, with the rapid development of the information age, users are more inclined to use various information platforms to query required information, such as whether they are suffering from diseases according to their own symptoms, and when a certain communicable disease (such as chickenpox) breaks out, there is bound to be more search traffic or attention. Therefore, in the embodiment, the daily public opinion factors of different regions in historical 20 years are selected as another part of the portrait data. The public opinion factors include, but are not limited to, chickenpox, redness and swelling, pruritus herpes, water herpes, etc.

The public opinion data includes at least one original entry (e.g., Baidu entry). Specifically, it is determined by an expert whether each original entry crawled is related to chickenpox based on the information contained in the original entry, so as to determine at least one entry that is truly related to chickenpox as the hit entry. Then, the public opinion factor is determined according to the determined hit entry. Each hit entry corresponds to a public opinion factor. The public opinion factor refers to at least one factor related to the preset keywords in the hit entry, such as chickenpox, redness and swelling, prurticant herpes, and water herpes.

At S30, medical data in historical unit time and a public opinion index corresponding to the hit entry are obtained, the public opinion index carrying a time label.

The medical data refers to the number of historical cases (i.e., label data) in historical unit time, for example, 20 years, of sentinel hospitals in different regions, that is provided by the Centers for Disease Control and Prevention. Understandably, the unit time is a time label, and may be customizable by the user, which is not limited here. In the embodiment, the unit time may be a day, a week, a month, a quarter, or a year, just to name a few.

In the embodiment, taking that the unit time is a week for example, specifically, the public opinion index corresponding to the hit entry in the unit time and the medical data are obtained. Each public opinion index carries the time label, and the time label refers to the time of publication of the hit entry.

At S40, the public opinion factor and the public opinion index carrying the time label are taken as first portrait data.

The first portrait data refers to taking the public opinion factor and the public opinion index carrying the time label as the feature data for model training. Specifically, when it is necessary to forecast whether a disease will break out in a certain future time interval, which may be one week, one month, one quarter, or one year, depending on the time interval of forecast, the processing of sample data will be different. Taking that the time interval is one week for example, part of portrait data may be set up by taking the public opinion factors (such as chickenpox, redness and swelling, and herpes) as column labels, and taking the public opinion indexes of the N-th week as row labels. The public opinion indexes of the N-th week include, but not limited to, an average public opinion index of the N-th week (that is, the average of the public opinion indexes of 7 days a week), the maximum public opinion index of the N-th week and the minimum public opinion index of the N-th week.

It is to be noted that the following table is a schematic diagram of the portrait data set up according to the public opinion factor in the embodiment. Understandably, the schematic diagram is illustrative and does not form a limit here.

| The public opinion | Redness and | | | |
|---|---|---|---|---|
| index of the N-th week | swelling | Chickenpox | Herpes | . . . |
| The public opinion | X₁ | X₂ | X₃ | . . . |
| index of the first week | | | | |
| The maximum public opinion | Y₁ | Y₂ | Y₃ | . . . |
| index of the first week | | | | |
| The minimum public opinion | Z₁ | Z₂ | Z₃ | . . . |
| index of the first week | | | | |
| . . . | . . . | . . . | . . . | . . . |
| The N-th week | . . . | . . . | . . . | . . . |

At S50, original sample data is obtained based on the first portrait data and the medical data.

Specifically, the first portrait data is taken as the feature data of model training, and the medical data is taken as the label data of model training, so as to obtain the original sample data.

At S60, the original sample data is cleaned to obtain sample data to be processed.

Specifically, because the original sample data may include a missing value or an abnormal value, in order to further ensure the accuracy of subsequent model forecast, it is necessary to clean the original sample data to ensure the quality of the sample data to be processed.

At S70, lag processing is performed on the sample data to be processed to obtain lag sample data.

The lag processing is a feature engineering method to collect more information by expanding a sample data set, that is, by augmenting a feature portrait. From the perspective of service logic, this is an effect of lag feature. Specifically, due to the different themes forecasted by some models, the corresponding sample data has a lag, such as the outbreak of disease or the data related to economy. In the embodiment, it is supposed that the theme of forecast is the forecast of chickenpox, and there is a lag in the outbreak of chickenpox, for example, a sudden rise in temperature and humid climate this week may not bring the outbreak of chickenpox this week, but the outbreak period will come next week, so it is necessary to performing lagging to the sample data to be processed to ensure the accuracy of subsequent model forecast. Specifically, n (which is generally 1 to 3) times of lag processing are performed on the sample data to be processed. If n is 1, lag processing is performed on the sample data to be processed, that is, the original data of the first week is taken as the data of the second week, the data of the second week is taken as the data of the third week, and so on, so as to obtain the lag sample data. If n is 2, the second lagging processing is performed based on the sample data obtained from the first lagging processing, so lag processing is performed on the sample data to be processed, that is, the original data of the first week is taken as the data of the third week, the data of the second week is taken as the data of the fourth week, and so on, so as to obtain the lag data; then, the lag data obtained each time is integrated to obtain the lag sample data and achieve the purpose of expanding the sample data set.

Finally, a concat function is used for combining the lag sample data obtained by multiple times of lag processing and the sample data to be processed into a data frame, that is, the lag sample data. The concat function is a function used for joining two or more arrays. The data frame is a two-dimensional data structure in which data is arranged in a table of rows and columns.

At S80, feature expansion is performed on the lag sample data to obtain target sample data.

Specifically, in order to expand the sample data set and further improve the accuracy of model forecast, in the embodiment, feature expansion is performed on the lag sample data to obtain the target sample data, so as to achieve the purpose of further expanding the sample data set.

At S90, an improved multi-granularity cascading random forest algorithm is used to train the target sample data to obtain a target forecast model. The improved multi-granularity cascading random forest algorithm includes a pooling layer which is used for retaining data features.

The improved multi-granularity cascading random forest algorithm is an algorithm that introduces the pooling idea of a convolutional neural network in a multi-granularity cascading random forest algorithm. The multi-granularity cascading random forest algorithm is a decision tree integration method that stacks multiple layers of random forests in a cascading way to obtain better feature representation and learning performance. The algorithm can achieve good performance without too much adjustment of super parameters.

Each layer in a multi-granularity cascading forest (Gcforest) is composed of several random forests. The random forest learns feature information of an input feature vector, and then inputs it to the next layer after processing. In order to enhance the generalization ability of the model, many different types of random forests are selected for each layer, which are respectively completely-random tree forests and random forests.

In the embodiment, first, according to the preset keywords, the crawler tool is used to crawl the public opinion data obtained by the third-party information platform, so as to determine at least one hit entry truly related to the forecast theme based on the public opinion data, and ensure the validity and accuracy of the public opinion factors obtained later. Then, the public opinion index and medical data corresponding to the hit entry in unit time is obtained. Finally, the public opinion factor and the public opinion index carrying the time label are taken as the original sample data, so that the model analyzes the public opinion data in the historical unit time, that is, 20 years. Then, the sample data to be processed is obtained by cleaning the original sample data, so as to ensure the quality of the sample data to be processed. Then, lag processing is performed on the sample data to be processed to obtain the lag sample data, so as to expand the sample data set. In addition, for the data with a lag, the effect of lag feature may be realized to ensure the accuracy of model forecast. Then, feature expansion is performed on the lag sample data to obtain the target sample data, so as to achieve the purpose of further expanding the sample data set and improving the accuracy of model forecast. Finally, the improved multi-granularity cascading random forest algorithm is used to train the target sample data to obtain the target forecast model, so as to obtain better feature representation and learning performance. Moreover, the algorithm may achieve good performance without too much adjustment of super parameters and ensure the accuracy of model forecast. In addition, the improved multi-granularity cascading random forest algorithm also includes a pooling layer to fully retain the data feature and further improve the accuracy of model forecast.

In an embodiment, before S10, the intelligent data analysis method further includes the following steps.

A meteorological factor and corresponding meteorological data are obtained.

Understandably, the embodiment may select different portrait data according to different forecast themes. In the embodiment, taking the forecast of chickenpox for example, because of the very close correlation between climatic conditions and chickenpox virus, daily meteorological factors over a 20-year history in different regions are selected as part of the portrait data. The meteorological factors include, but not limited to, diurnal temperature, diurnal atmospheric pressure, diurnal precipitation, humidity, light intensity, and wind power in different regions.

The meteorological factor and the corresponding meteorological data are taken as second portrait data.

The second portrait data refers to taking the meteorological factor and the corresponding meteorological data as the feature data of model training. Specifically, the way of setting up the portrait data for the meteorological factor is consistent with S40, that is, the second portrait data may be set up by taking the meteorological factors as the column labels, and taking the meteorological conditions in the N-th week as the row labels. The meteorological conditions in the N-th week include, but not limited to, the average meteorological condition in the N-th week (such as the average precipitation), the maximum meteorological condition in the N-th week (such as the maximum precipitation) and the minimum meteorological condition in the N-th week (such as the minimum precipitation).

Correspondingly, S50 in which the original sample data is obtained based on the first portrait data and the medical data includes the following steps.

The first portrait data, the second portrait data and the medical data are taken as the original sample data.

In the embodiment, through the idea of the meteorological conditions combined with the mass dissemination of public opinion data, a disease outbreak period may be effectively forecasted and the accuracy of model forecast may be improved.

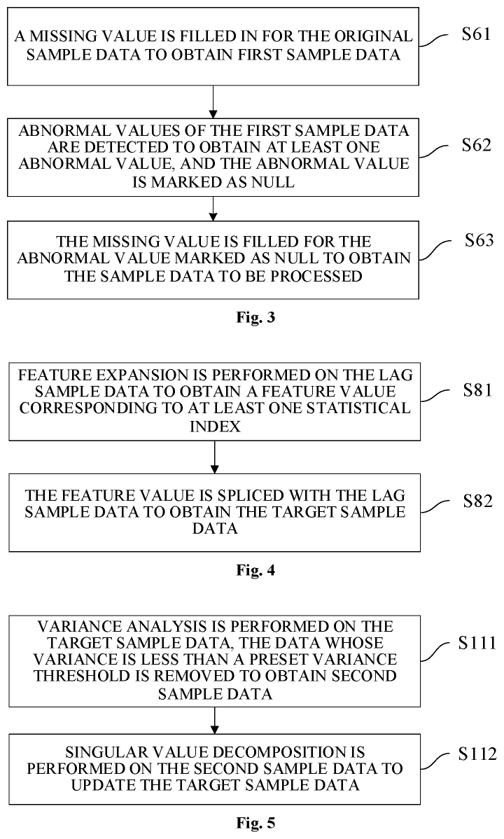

In an embodiment, as shown in FIG. 3, S60 in which the original sample data is cleaned to obtain the sample data to be processed specifically includes the following steps.

At S61, a missing value is filled in for the original sample data to obtain first sample data.

The methods for filing in the missing value include, but not limited to, mean filling, mode filling, median filling, expected value maximization method, multiple filling, and k-means clustering methods. Specifically, taking the k-means clustering method for filling as an example, the portrait data where the missing value is located is clustered, and the missing value is filled with the mean value of the clusters.

At S62, abnormal values of the first sample data are detected to obtain at least one abnormal value, and the abnormal value is marked as null.

At S63, the missing value is filled for the abnormal value marked as null to obtain the sample data to be processed.

Specifically, the detection of abnormal value includes, but is not limited to, the use of statistical variable analysis (such as box-plot analysis, mean value analysis, maximum and minimum analysis, and the 3σ rule), distance-based methods, density-based outlier detection, and isolation forest. In the embodiment, taking the 3σ rule as an example, if the data obeys a normal distribution, in the 3σ rule, the abnormal value is defined as the value that is more than three standard deviations from the mean value in a set of measured values, that is because the probability of occurrence of a value outside the mean value 3σ is less than 0.003 under the assumption of normal distribution, that is, the data exceeding μ+3σ and the data not exceeding μ−3σ are taken as the abnormal values.

Specifically, because the abnormal value corresponding to the sample data is not necessarily unnecessary, if the sample data corresponding to the abnormal value is deleted directly, it will lead to missing features in the sample data and affect the quality of the sample data, thus affecting the accuracy of model forecast. Therefore, in the embodiment, the abnormal value will be deleted and marked as null, and then the abnormal value marked as null will be filled with the missing value again to obtain the sample data to be processed. In the embodiment, by filling in the missing value of the abnormal value marked as null, the sample data to be processed is obtained, so as to avoid directly removing the sample data corresponding to the abnormal value, which results in the lack of this part of features of the sample data and affects the accuracy of model forecast.

In the embodiment, the first sample data is obtained by filling in the missing value of the original sample data, and then the abnormal values of the first sample data is detected to obtain at least one abnormal value, so as to achieve the purpose of cleaning data and ensure the quality of the sample data by processing the abnormal value and the missing value in the sample data. Then, the obtained abnormal value is marked as null, so that the abnormal value marked as null is filled with the missing value again to obtain the sample to be processed. By filling the original sample data with the missing value twice, the quality and standardization of the sample data can be guaranteed and the accuracy of model forecast can be improved.

In an embodiment, as shown in FIG. 4, S80 in which feature expansion is performed on the lag sample data to obtain the target sample data specifically includes the following steps.

At S81, feature expansion is performed on the lag sample data to obtain a feature value corresponding to at least one statistical index.

At S82, the feature value is spliced with the lag sample data to obtain the target sample data.

The statistical indexes include, but not limited to, the maximum value, the minimum value, the mean value, and a standard deviation corresponding to each row of data. Each statistical index is added to the lag sample data as a new column to expand the data set, increase a feature portrait to collect more feature information, and improve the accuracy of model forecast. Understandably, the lag sample data is a matrix, and the feature value is spliced with the lag sample data to obtain the target sample data, that is, N columns are added to the sample matrix, N being the number of statistical indexes (such as the maximum value, the minimum value, and the mean value corresponding to each row of data), and the maximum value, the minimum value, and the mean value corresponding to each row of data are the feature values.

In the embodiment, the feature value corresponding to at least one statistical index is obtained by performing feature expansion on the lag sample data. The feature value is spliced with the lag sample data to obtain the target sample data, so as to expand the data set, increase the feature portrait to collect more feature information, and improve the accuracy of model forecast.

In an embodiment, as shown in FIG. 5, after S80, the intelligent data analysis method further includes the following steps.

At S111, variance analysis is performed on the target sample data, the data whose variance is less than a preset variance threshold is removed to obtain second sample data.

At S112, singular value decomposition is performed on the second sample data to update the target sample data.

Specifically, because sometimes too much data is not a good thing, a large amount of data in data analysis applications may lead to worse performance. Therefore, it is necessary to filter the target sample data to remove redundant data, so as to ensure the loss of data information as little as possible while reducing the number of data columns.

Variance analysis refers to the analysis based on the variance of the data column to remove the sequence with too small variance (that is, less than the preset variance threshold) and obtain the second sample data. Specifically, the size of variance describes the amount of information in a variable, and the sequence with too small variance is considered to contain little information, so all the data columns with small variance are removed to achieve the effect of data dimension reduction, reduce data processing capacity, and improve the efficiency of subsequent model training.

Specifically, there are many features included in the target sample data, but some features have little influence on the accuracy of the model forecast, or it may be considered that the features that are too correlated may be replaced equally, so redundant variables may be removed to achieve the purpose of data dimension reduction and save the time of model training. Specifically, when the variance analysis is adopted, the data columns whose variance is less than the preset variance threshold are removed, so the accuracy of the variance analysis depends on the preset variance threshold. Therefore, in order to further remove redundant data and ensure the loss of data information as little as possible, in the embodiment, it is also necessary to perform singular value decomposition to the second sample data, so as to remove the redundant data, achieve the purpose of data compression, and ensure the quality of the target sample data.

In the embodiment, by performing the variance analysis to the target sample data and removing the data whose variance is less than the preset variance threshold, the second sample data is obtained, so as to remove the redundant data, ensure the loss of data information as little as possible while reducing the number of data columns, and save the time of model training. Then, singular value decomposition is performed on the second sample data, and the target sample data is updated, so as to further remove the redundant data and ensure the quality of the target sample data.

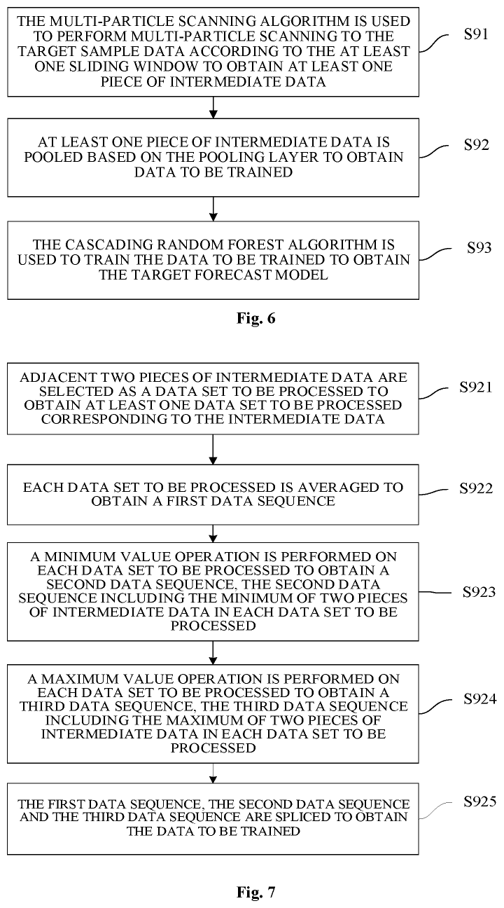

In an embodiment, the improved multi-granularity cascading random forest algorithm includes the multi-particle scanning algorithm and the cascading random forest algorithm. The multi-particle scanning algorithm corresponds to at least one sliding window. As shown in FIG. 6, S90 specifically includes the following steps.

At S91, the multi-particle scanning algorithm is used to perform multi-particle scanning to the target sample data according to the at least one sliding window to obtain at least one piece of intermediate data.

The multi-particle scanning algorithm refers to using the sliding window to scan the target sample data to obtain at least one piece of intermediate data. In the embodiment, the sliding windows of different dimensions may be set. Understandably, the sliding window may be an i*j window. For example, if the row label of the target sample data is the i-th week, then the window_size of the sliding widow may be 2 (every 2 weeks), 4 (every month), 12 (every quarter), and so on. It is to be noted that the sliding window may scan at least one feature portrait, that is, may scan every column, every two columns, and every j columns, so as to maximize the search for the intrinsic correlation between features and tag set, features and features.

At S92, at least one piece of intermediate data is pooled based on the pooling layer to obtain data to be trained.

In the embodiment, the data to be trained is obtained by pooling the at least one piece of intermediate data, so as to achieve the purpose of dimension reduction of the data, reduce the amount of computation, and improve the efficiency of model training.

At S93, the cascading random forest algorithm is used to train the data to be trained to obtain the target forecast model.

Specifically, based on the idea of neural network integration, the multi-granularity cascading random forest algorithm takes the label column cforestᵢ obtained from the i-th complete-random tree forest and the label column rforestᵢ obtained from the random forest as portrait columns that are continuously added to the target sample data, so as to further expand features and finally obtain the following feature portrait [orgf₁, orgf₂, . . . , orgfₙ, cforest₁, rforest₁, . . . , cforestₖ, rforestₖ], where orgf is the target sample data. Finally, the feature portrait is input into the final m (m is generally 3 to 5, 3 for general order of magnitude, 3 to 4 for ten million order of magnitude, and 4 to 5 for over ten million order of magnitude) random forecasts for forecasting, and the final Max value is taken as the final forecast probability value.

Specifically, the obtained data to be trained is input into the cascading forest for training. For example, the sliding windows of three dimensions are used in the embodiment. Firstly, the sliding window of the first dimension is used for scanning to obtain a feature vector, and the original feature vector is input into the complete-random tree forest and the random forest to respectively obtain two forecast sequences (that is, cforestᵢ and rforestᵢ); and then the two forecast sequences are spliced to obtain a first feature vector, and the original feature vector is input into the cascading forest of the first layer for training to obtain a first forecast sequence. Then, the obtained first forecast sequence is spliced with the first feature vector to obtain a second feature vector as input data of the cascading forest of the second layer; a second forecast sequence trained by the cascading forest of the second layer is spliced with a third feature vector obtained by the sliding window of the second dimension (by means of the same method as the first feature vector) as input data of the cascading forest of the third layer; a third forecast sequence trained by the cascading forest of the third layer is spliced with a fourth feature vector obtained by the sliding window of the third dimension as the input of the next layer. The above process is repeated until convergence and the target forecast model is obtained.

In the embodiment, by using the multi-particle scanning algorithm to perform multi-particle scanning to the target sample data based on the at least one sliding window, at least one piece of intermediate data is obtained, so as to maximize the search of internal correlation between the feature and the label set and between the features. Then, in combination with the pooling layer, at least one piece of intermediate data is pooled to obtain the data to be trained, so as to combine machine learning with neural network idea to obtain more information that cannot be obtained intuitively, thus enriching the model, and further improving the accuracy of model forecast.

In an embodiment, as shown in FIG. 7, S92 in which at least one piece of intermediate data is pooled based on the pooling layer to obtain the data to be trained specifically includes the following steps.

At S921, adjacent two pieces of intermediate data are selected as a data set to be processed to obtain at least one data set to be processed corresponding to the intermediate data.

At S922, each data set to be processed is averaged to obtain a first data sequence.

At S923, a minimum value operation is performed on each data set to be processed to obtain a second data sequence, the second data sequence including the minimum of two pieces of intermediate data in each data set to be processed.

At S924, a maximum value operation is performed on each data set to be processed to obtain a third data sequence, the third data sequence including the maximum of two pieces of intermediate data in each data set to be processed.

At S925, the first data sequence, the second data sequence and the third data sequence are spliced to obtain the data to be trained.

Specifically, from the perspective of service logic, the model forecast requires more linear or nonlinear methods to distort the data in space, so as to obtain more information that cannot be obtained intuitively to enrich the model. Therefore, in the embodiment, three pooling methods are used to pool at least one piece of intermediate data, and then the results obtained by pooling in each method are integrated to obtain the data to be trained, so as to obtain more information that cannot be obtained intuitively to enrich the model, and fully retain the data features. Assuming that the middle is a certain column of portrait data Feature: f₁, f₂, f₃, f₄, f₅, . . . fₙ in the intermediate data, then at least one piece of intermediate data is pooled in the following three pooling methods.
Feature_new_1: (f₁+f₂)/2, (f₂+f₃)/2, . . . , (fₙ₋₁+fₙ)/2
Feature_new_2: max (f₁, f₂), max (f₂, f₃), . . . , max (fₙ₋₁, fₙ)
Feature_new_3: min(f₁, f₂), min(f₂, f₃), . . . , min(fₙ₋₁, fₙ)

In the embodiment, at least one piece of intermediate data is pooled in three pooling methods, and then the results obtained by pooling in each method are integrated to obtain the data to be trained, so as to fully retain the data features, ensure the quality of sample data, and improve the accuracy of model forecast.

It should be understood that, in the above embodiments, a magnitude of a sequence number of each step does not mean an execution sequence and the execution sequence of each process should be determined by its function and an internal logic and should not form any limit to an implementation process of the embodiments of the disclosure.

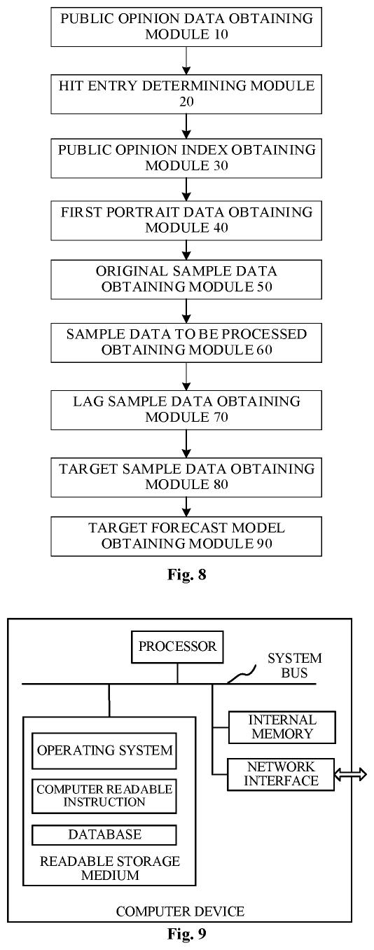

In an embodiment, an intelligent data analysis device is provided. The intelligent data analysis device corresponds to the intelligent data analysis method in the above embodiment. As shown in FIG. 8, the intelligent data analysis device includes a public opinion data obtaining module 10, a hit entry determining module 20, a public opinion index obtaining module 30, a first portrait data obtaining module 40, an original sample data obtaining module 50, a sample data to be processed obtaining module 60, a lag sample data obtaining module 70, a target sample data obtaining module 80 and a target forecast model obtaining module 90. Each functional module is described in detail below.

The public opinion data obtaining module 10 is configured to, according to the preset keywords, use the crawler tool to crawl the public opinion data obtained by the third-party information platform.

The hit entry determining module 20 is configured to determine at least one hit entry based on the public opinion data, the hit entry corresponding to the public opinion factor.

The public opinion index obtaining module 30 is configured to obtain the medical data in the historical unit time and the public opinion index corresponding to the hit entry, the public opinion index carrying the time label.

The first portrait data obtaining module 40 is configured to take the public opinion factor and the public opinion index carrying the time label as the first portrait data.

The original sample data obtaining module 50 is configured to obtain the original sample data based on the first portrait data and the medical data.

The sample data to be processed obtaining module 60 is configured to clean the original sample data to obtain the sample data to be processed.

The lag sample data obtaining module 70 is configured to perform lag processing on the sample data to be processed to obtain the lag sample data.

The target sample data obtaining module 80 is configured to perform feature expansion on the lag sample data to obtain the target sample data.

The target forecast model obtaining module 90 is configured to use the improved multi-granularity cascading random forest algorithm to train the target sample data to obtain the target forecast model, the improved multi-granularity cascading random forest algorithm including the pooling layer which is used for retaining the data features.

Specifically, the sample data to be processed obtaining module includes a first sample data obtaining unit, an abnormal value obtaining unit and a sample data to be processed obtaining unit.

The first sample data obtaining unit is configured to fill in the missing value for the original sample data to obtain first sample data.

The abnormal value obtaining unit is configured to detect the abnormal values of the first sample data to obtain at least one abnormal value, and mark the abnormal value as null.

The sample data to be processed obtaining unit is configured to fill in the missing value for the abnormal value marked as null to obtain the sample data to be processed.

Specifically, the target sample data obtaining module includes a feature value obtaining unit and a target sample data obtaining unit.

The feature value obtaining unit is configured to perform feature expansion the lag sample data to obtain the feature value corresponding to at least one statistical index.

The target sample data obtaining unit is configured to splice the feature value with the lag sample data to obtain the target sample data.

Specifically, the intelligent data analysis device includes a second sample data obtaining unit and a target sample data updating unit.

The second sample data obtaining unit is configured to perform variance analysis to the target sample data, remove the data whose variance is less than a preset variance threshold to obtain second sample data.

The target sample data updating unit is configured to perform singular value decomposition to the second sample data to update the target sample data.

Specifically, the improved multi-granularity cascading random forest algorithm includes the multi-particle scanning algorithm and the cascading random forest algorithm. The multi-particle scanning algorithm corresponds to at least one sliding window. The target forecast model obtaining module includes an intermediate data obtaining unit, a data to be trained obtaining unit and a target forecast model obtaining unit.

The intermediate data obtaining unit is configured to use the multi-particle scanning algorithm to perform multi-particle scanning to the target sample data according to the at least one sliding window to obtain at least one piece of intermediate data.

The data to be trained obtaining unit is configured to pool at least one piece of intermediate data based on the pooling layer to obtain the data to be trained.

The target forecast model obtaining unit is configured to use the cascading random forest algorithm to train the data to be trained to obtain the target forecast model.

Specifically, the data to be trained obtaining unit includes a data set to be processed obtaining subunit, a first data sequence obtaining subunit, a second data sequence obtaining subunit, a third data sequence obtaining subunit and a data to be trained obtaining subunit.

The data set to be processed obtaining subunit is configured to select adjacent two pieces of intermediate data as a data set to be processed to obtain at least one data set to be processed corresponding to the intermediate data.

The first data sequence obtaining subunit is configured to average each data set to be processed to obtain a first data sequence.

The second data sequence obtaining subunit is configured to perform a minimum value operation to each data set to be processed to obtain a second data sequence, the second data sequence including the minimum of two pieces of intermediate data in each data set to be processed.

The third data sequence obtaining subunit is configured to perform a maximum value operation to each data set to be processed to obtain a third data sequence, the third data sequence including the maximum of two pieces of intermediate data in each data set to be processed.

The data to be trained obtaining subunit is configured to splice the first data sequence, the second data sequence and the third data sequence to obtain the data to be trained.

For specific descriptions of the intelligent data analysis device, please refer to the descriptions of the intelligent data analysis method mentioned above, which will not be repeated here. Each module in the intelligent data analysis device may be realized in whole or in part by software, hardware, and their combination. Each above module may be embedded in or independent of a processor in a computer device in the form of hardware, or stored in a memory in the computer device in the form of software, so that the processor may call and perform the operation corresponding to each module above.

In an embodiment, a computer device is provided. The computer device may be a server, and its internal structure may be shown in FIG. 9. The computer device includes a processor, a memory, a network interface, and a database connected through a system bus. The processor of the computer device is used to provide computing and control capabilities. The memory of the computer device includes a readable storage medium and an internal memory. The readable storage medium stores an operating system, a computer readable instruction, and a database. The internal memory provides an environment for the operation of the operating system and the computer readable instruction in the readable storage medium. The database of the computer device is used to store the data, such as the target sample data, generated or acquired during the execution of the intelligent data analysis method. The network interface of the computer device is used to communicate with an external terminal through a network connection. The computer readable instruction, when executed by the processor, implements an intelligent data analysis method.

In an embodiment, a computer device is provided, which includes: a memory, a processor, and a computer readable instruction stored in the memory and capable of running on the processor. The processor, when executing the computer readable instruction, implements the steps of the intelligent data analysis method in the above embodiment, such as S10 to S90 shown in FIG. 2 or the steps shown in FIG. 3 to FIG. 7. Or, the processor, when executing the computer readable instruction, realizes the functions of each module/unit in the embodiment of the intelligent data analysis device, such as the functions of each module/unit shown in FIG. 8, which will not be described here to avoid repetition.

In an embodiment, one or more readable storage media storing a computer readable instruction are provided. The computer-readable storage medium stores a computer readable instruction. The computer readable instruction, when executed by one or more processors, enables the one or more processors to implement the steps of the intelligent data analysis method in the above embodiment, such as S10 to S90 shown in FIG. 2 or the steps shown in FIG. 3 to FIG. 7, which will not be described here to avoid repetition. Or, the computer readable instruction, when executed by the processor, realizes the functions of each module/unit in the embodiment of the intelligent data analysis device, such as the functions of each module/unit shown in FIG. 8, which will not be described here to avoid repetition. The readable storage medium in the embodiment includes a non-volatile readable storage medium and a volatile readable storage medium.

Those of ordinary skill in the art may understand that all or part of flows of the method in the above embodiments may be completed by related hardware instructed by a computer readable instruction. The computer readable instruction may be stored in a non-volatile computer readable storage medium. When executed, the computer readable instruction may include the flows in the embodiments of the method. Any reference to memory, storage, database, or other media used in each embodiment provided in the application may include non-volatile and/or volatile memories. The non-volatile memories may include a Read-Only Memory (ROM), a Programmable Read-Only Memory (PROM), an Electrically Programmable Read-Only Memory (EPROM), an Electrically Erasable Programmable Read-Only Memory (EEPROM) or a flash memory. The volatile memories may include a Random Access Memory (RAM) or an external cache memory. As an illustration rather than a limitation, the RAM is available in many forms, such as Static RAM (SRAM), Dynamic RAM (DRAM), Synchronous DRAM (SDRAM), Double Data Rate SDRAM (DDRAM), Enhanced SDRAM (ESDRAM), Synch-link DRAM (SLDRAM), Rambus Direct RAM (RDRAM), Direct Memory Bus Dynamic RAM (DRDRAM), and Memory Bus Dynamic RAM (RDRAM).

Those of ordinary skill in the art may clearly understand that for the convenience and simplicity of description, illustration is given only based on the division of the above functional units and modules. In practical applications, the above functions may be allocated to different functional units and modules for realization according to needs, that is, the internal structure of the device is divided into different functional units or modules to realize all or part of the functions described above.

The above embodiments are only used for illustrating, but not limiting, the technical solutions of the disclosure. Although the disclosure is elaborated referring to the above embodiments, those of ordinary skill in the art should understand that they may still modify the technical solutions in each above embodiment, or equivalently replace a part of technical features; but these modifications and replacements do not make the nature of the corresponding technical solutions depart from the spirit and scope of the technical solutions in each embodiment of the disclosure, and these modifications and replacements should be included in the scope of protection of the disclosure.

What is claimed is:

1. An intelligent data analysis method, comprising:
according to preset keywords, using a crawler tool to crawl public opinion data obtained by a third-party information platform;
determining at least one hit entry based on the public opinion data, wherein the hit entry corresponds to a public opinion factor;
obtaining medical data in historical unit time and a public opinion index corresponding to the at least one hit entry, wherein the public opinion index carries a time label;
taking the public opinion factor and the public opinion index that carries the time label as first portrait data;
obtaining original sample data based on the first portrait data and the medical data;
cleaning the original sample data to obtain sample data to be processed;
performing lag processing on the sample data to be processed to obtain lag sample data;
performing feature expansion on the lag sample data to obtain target sample data; and
using an improved multi-granularity cascading random forest algorithm to train the target sample data to obtain a target forecast model, wherein the improved multi-granularity cascading random forest algorithm comprises a pooling layer which is used for retaining data features.

2. The intelligent data analysis method as claimed in claim 1, wherein before according to the preset keywords, using the crawler tool to crawl the public opinion data obtained by the third-party information platform, the intelligent data analysis method further comprises:
obtaining a meteorological factor and corresponding meteorological data; and
taking the meteorological factor and the corresponding meteorological data as second portrait data;
wherein obtaining the original sample data based on the first portrait data and the medical data comprises:
taking the first portrait data, the second portrait data, and the medical data as the original sample data.

3. The intelligent data analysis method as claimed in claim 1, wherein cleaning the original sample data to obtain the sample data to be processed comprises:
filling in a missing value for the original sample data to obtain first sample data;
detecting abnormal values of the first sample data to obtain at least one abnormal value, and marking the abnormal value as null; and
filling in the missing value for the abnormal value marked as null to obtain the sample data to be processed.

4. The intelligent data analysis method as claimed in claim 1, wherein performing feature expansion on the lag sample data to obtain the target sample data comprises:
performing feature expansion on the lag sample data to obtain a feature value corresponding to at least one statistical index; and
splicing the feature value with the lag sample data to obtain the target sample data.

5. The intelligent data analysis method as claimed in claim 1, wherein after obtaining the target sample data, the intelligent data analysis method comprises:
performing variance analysis on the target sample data and removing the data whose variance is less than a preset variance threshold to obtain second sample data; and
performing singular value decomposition on the second sample data to update the target sample data.

6. The intelligent data analysis method as claimed in claim 1, wherein the improved multi-granularity cascading random forest algorithm comprises a multi-particle scanning algorithm and a cascading random forest algorithm and the multi-particle scanning algorithm corresponds to at least one sliding window; and
wherein using the improved multi-granularity cascading random forest algorithm to train the target sample data to obtain the target forecast model comprises:
using the multi-particle scanning algorithm to perform multi-particle scanning on the target sample data according to the at least one sliding window to obtain at least one piece of intermediate data;
pooling the at least one piece of intermediate data based on the pooling layer to obtain data to be trained; and
using the cascading random forest algorithm to train the data to be trained to obtain the target forecast model.

7. The intelligent data analysis method as claimed in claim 6, wherein pooling the at least one piece of intermediate data based on the pooling layer to obtain the data to be trained comprises:
selecting adjacent two pieces of intermediate data as a data set to be processed to obtain at least one data set to be processed corresponding to the intermediate data;
averaging each data set to be processed to obtain a first data sequence;
performing a minimum value operation on each data set to be processed to obtain a second data sequence, wherein the second data sequence comprises a minimum of two pieces of intermediate data in each data set to be processed;
performing a maximum value operation on each data set to be processed to obtain a third data sequence, wherein the third data sequence comprises a maximum of two pieces of intermediate data in each data set to be processed; and
splicing the first data sequence, the second data sequence, and the third data sequence to obtain the data to be trained.

8. A computer device, comprising:
a memory, a processor, and a computer readable instruction stored in the memory and capable of running on the processor, wherein the processor, when executing the computer readable instruction, is configured to perform:
according to preset keywords, using a crawler tool to crawl public opinion data obtained by a third-party information platform;
determining at least one hit entry based on the public opinion data, wherein the hit entry corresponds to a public opinion factor;
obtaining medical data in historical unit time and a public opinion index corresponding to the at least one hit entry, wherein the public opinion index carries a time label;
taking the public opinion factor and the public opinion index that carries the time label as first portrait data;
obtaining original sample data based on the first portrait data and the medical data;
cleaning the original sample data to obtain sample data to be processed;
performing lag processing on the sample data to be processed to obtain lag sample data;
performing feature expansion on the lag sample data to obtain target sample data; and
using an improved multi-granularity cascading random forest algorithm to train the target sample data to obtain a target forecast model, wherein the improved multi-granularity cascading random forest algorithm comprises a pooling layer which is used for retaining data features.

9. The computer device as claimed in claim 8, wherein the processor is further configured to perform:
before according to the preset keywords, using the crawler tool to crawl the public opinion data obtained by the third-party information platform:
obtaining a meteorological factor and corresponding meteorological data; and
taking the meteorological factor and the corresponding meteorological data as second portrait data;
wherein obtaining the original sample data based on the first portrait data and the medical data comprises:
taking the first portrait data, the second portrait data and the medical data as the original sample data.

10. The computer device as claimed in claim 8, wherein to perform cleaning the original sample data to obtain the sample data to be processed, the processor is configured to perform:
filling in a missing value for the original sample data to obtain first sample data;
detecting abnormal values of the first sample data to obtain at least one abnormal value, and marking the abnormal value as null; and
filling in the missing value for the abnormal value marked as null to obtain the sample data to be processed.

11. The computer device as claimed in claim 8, wherein to perform performing feature expansion to the lag sample data to obtain the target sample data, the processor is configured to perform:
performing feature expansion on the lag sample data to obtain a feature value corresponding to at least one statistical index; and
splicing the feature value with the lag sample data to obtain the target sample data.

12. The computer device as claimed in claim 8, wherein the processor is further configured to perform:
after obtaining the target sample data:
performing variance analysis on the target sample data and removing the data whose variance is less than a preset variance threshold to obtain second sample data; and
performing singular value decomposition on the second sample data to update the target sample data.

13. The computer device as claimed in claim 8, wherein the improved multi-granularity cascading random forest algorithm comprises a multi-particle scanning algorithm and a cascading random forest algorithm and the multi-particle scanning algorithm corresponds to at least one sliding window;
wherein to perform using the improved multi-granularity cascading random forest algorithm to train the target sample data to obtain the target forecast model, the processor is configured to perform:
using the multi-particle scanning algorithm to perform multi-particle scanning on the target sample data according to the at least one sliding window to obtain at least one piece of intermediate data;
pooling the at least one piece of intermediate data based on the pooling layer to obtain data to be trained; and
using the cascading random forest algorithm to train the data to be trained to obtain the target forecast model.

14. The computer device as claimed in claim 13, wherein to perform pooling the at least one piece of intermediate data based on the pooling layer to obtain the data to be trained, the processor is configured to perform:
selecting adjacent two pieces of intermediate data as a data set to be processed to obtain at least one data set to be processed corresponding to the intermediate data;
averaging each data set to be processed to obtain a first data sequence;
performing a minimum value operation on each data set to be processed to obtain a second data sequence, wherein the second data sequence comprises a minimum of two pieces of intermediate data in each data set to be processed;
performing a maximum value operation on each data set to be processed to obtain a third data sequence, wherein the third data sequence comprises a maximum of two pieces of intermediate data in each data set to be processed; and
splicing the first data sequence, the second data sequence and the third data sequence to obtain the data to be trained.

15. A readable storage media that stores a computer readable instruction, wherein the computer readable instruction, when executed by one or more processors, enables the one or more processors to perform:
according to preset keywords, using a crawler tool to crawl public opinion data obtained by a third-party information platform;
determining at least one hit entry based on the public opinion data, wherein the hit entry corresponds to a public opinion factor;
obtaining medical data in historical unit time and a public opinion index corresponding to the at least one hit entry, wherein the public opinion index carries a time label;
taking the public opinion factor and the public opinion index that carries the time label as first portrait data;
obtaining original sample data based on the first portrait data and the medical data;
cleaning the original sample data to obtain sample data to be processed;
performing lag processing on the sample data to be processed to obtain lag sample data;
performing feature expansion on the lag sample data to obtain target sample data; and
using an improved multi-granularity cascading random forest algorithm to train the target sample data to obtain a target forecast model, wherein the improved multi-granularity cascading random forest algorithm comprises a pooling layer which is used for retaining data features.

16. The readable storage media as claimed in claim 15, wherein the computer readable instruction, when executed by the one or more processors, enables the one or more processors to further perform:
before according to the preset keywords, using the crawler tool to crawl the public opinion data obtained by the third-party information platform:
obtaining a meteorological factor and corresponding meteorological data; and
taking the meteorological factor and the corresponding meteorological data as second portrait data;
wherein obtaining the original sample data based on the first portrait data and the medical data comprises:
taking the first portrait data, the second portrait data and the medical data as the original sample data.

17. The readable storage media as claimed in claim 15, wherein to perform cleaning the original sample data to obtain the sample data to be processed, the computer readable instruction, when executed by the one or more processors, enables the one or more processors to perform:
filling in a missing value for the original sample data to obtain first sample data;
detecting abnormal values of the first sample data to obtain at least one abnormal value, and marking the abnormal value as null; and
filling in the missing value for the abnormal value marked as null to obtain the sample data to be processed.

18. The readable storage media as claimed in claim 15, wherein to perform performing feature expansion on the lag sample data to obtain the target sample data, the computer readable instruction, when executed by the one or more processors, enables the one or more processors to perform:
performing feature expansion on the lag sample data to obtain a feature value corresponding to at least one statistical index; and
splicing the feature value with the lag sample data to obtain the target sample data.

19. The readable storage media as claimed in claim 15, wherein the improved multi-granularity cascading random forest algorithm comprises a multi-particle scanning algorithm and a cascading random forest algorithm and the multi-particle scanning algorithm corresponds to at least one sliding window;
wherein to perform using the improved multi-granularity cascading random forest algorithm to train the target sample data to obtain the target forecast model, the computer readable instruction, when executed by the one or more processors, enables the one or more processors to perform:
using the multi-particle scanning algorithm to perform multi-particle scanning on the target sample data according to the at least one sliding window to obtain at least one piece of intermediate data;
pooling the at least one piece of intermediate data based on the pooling layer to obtain data to be trained; and
using the cascading random forest algorithm to train the data to be trained to obtain the target forecast model.

20. The readable storage media as claimed in claim 19, wherein to perform pooling the at least one piece of intermediate data based on the pooling layer to obtain the data to be trained, the computer readable instruction, when executed by the one or more processors, enables the one or more processors to perform:
selecting adjacent two pieces of intermediate data as a data set to be processed to obtain at least one data set to be processed corresponding to the intermediate data;
averaging each data set to be processed to obtain a first data sequence;
performing a minimum value operation on each data set to be processed to obtain a second data sequence, wherein the second data sequence comprises a minimum of two pieces of intermediate data in each data set to be processed;
performing a maximum value operation on each data set to be processed to obtain a third data sequence, wherein the third data sequence comprises a maximum of two pieces of intermediate data in each data set to be processed; and
splicing the first data sequence, the second data sequence and the third data sequence to obtain the data to be trained.
